(19) United States
(12) Patent Application Publication
(10) Pub. No.: US 2016/0058817 A1
(43) Pub. Date: Mar. 3, 2016

(54) Treatment of precancerous lesions with Sanguinaria Canadensis Extract/Compounded Extract

(71) Applicants: Stephen K. Richardson, Tallahassee, FL (US); Albert Assad, Bound Brook, NJ (US)

(72) Inventors: Stephen K. Richardson, Tallahassee, FL (US); Albert Assad, Bound Brook, NJ (US)

(21) Appl. No.: 14/468,716

(22) Filed: Aug. 26, 2014

(51) Int. Cl. A61K 36/66; A61K 45/06

(52) U.S. Cl. CPC A61K 36/66; A61K 45/06

(57) ABSTRACT

The present invention relates to a novel extract of the plant Sanguinaria Canadensis (SC) in combination with steroids and/or non-steroidal anti-inflammatory compounds, methods of preparation thereof, and to its use. More specifically, the invention relates to the preparation of a solubilized form of SC consisting of its isoquinolone alkaloids, sanguinarine and chelerythrine, compounded with topical steroids (hydrocortisone, triamcinolone acetonide, betamethasone, betamethasone diproprionate, methylprednisolone acetonate types, et al) and/or non-steroidal anti-inflammatory compounds.

This application claims priority to U.S. patent application No. 61/870,227, filed Aug. 26, 2013, the contents of which, in part, are incorporated herein.

### FIELD OF THE INVENTION

This invention relates to a novel compound of SC, methods of preparation thereof, and its use.

### BACKGROUND OF THE INVENTION

Sanguinarine (S) and chelerythrine (C) derived from the plant SC represent a small class of isoquinolone alkaloids called natural quaternary benzo[c]phenanthridine alkaloids (QBAs). Their most common and richest natural sources are the plants Sanguinaria Canadensis (SC), Dicranostigma lacucoides, Chelidonium majus, Macleaya, Bocconia species from the Papveraceae family and Rutaceae.

SC extracts have been reported to have anti-microbial and anti-neoplastic properties. Numerous published in-vitro and in-vivo studies have demonstrated targeted apoptosis of human cancer cells induced by SC extracts. In addition, SC extracts have also been shown to induce the proliferation of human peripheral blood mononuclear cells and their production of pro-inflammatory Th-1 cytokines (which have proven anti-tumor properties).

There are no clinical trials evaluating the efficacy of topical formulations containing SC extracts for cutaneous disease. Current literature (case studies) reports variable clinical responses when such formulations have been used for the treatment of cutaneous malignancies with mild to severe adverse effects noted.

We have monitored two patients treated with a topical formulation containing SC, both of whom exhibited a complete clinical response to treatment of pre-cancerous lesions (actinic keratoses) and skin cancer (squamous cell carcinoma) with biopsy proven histologic clearance.

SC extracts are commercially available in a multiple formulations, however, none combining it with a steroid ingredient or non-steroidal anti-inflammatory ingredient to potentially enhance its efficacy, decrease inflammation and the risk of scarring.

### REFERENCES (INCORPORATED HEREIN BY REFERENCE)

U.S. Pat. No. 4,515,779 issued to Elliott on May 7, 1985.

### SUMMARY OF THE INVENTION

This invention is directed to a novel formulation of SC extracts compounded with different topical steroids (hydrocortisone, triamcinolone acetonide, betamethasone, betamethasone diproprionate, methylprednisolone acetate types, et al) and/or non-steroidal anti-inflammatory compounds.

### DESCRIPTION OF THE INVENTION

This present invention relates to a novel compound consisting of SC extract in combination with a steroid ingredient and/or non-steroidal anti-inflammatory compound. Current preparations of SC extracts vary in composition and none incorporate a steroid or non-steroidal anti-inflammatory drug (NSAID). Most cutaneous adverse effects related to commercially available topical SC medicaments stem from severe, uncontrolled inflammation and subsequent scarring. The addition of a steroid 0.01% to 10% and/or NSAID component 0.01% to 10% would limit the extent of inflammation, thus decreasing the risk for scarring among other potential adverse effects (e.g., severe ulceration, bleeding, infection), and potentially enhance anti-tumor activity.

Suspensions can be made from the proposed invention that may be used for aerosols, or processed into other pharmaceutical formulations such as creams, gels, ointments, or other topical applications.

In general, the proposed invention is dispersed in a vehicle (water or any other suitable vehicle where it is maintained as a suspension which has viscosity suitable for delivery and for preventing the active substance from settling during storage). The vehicle may also be solvent mixtures.

Further additives may be stabilizers, emulsifiers, penetration enhancers, gelling agents and other components commonly used in dermal formulations, e.g., antioxidants, preservatives, pigments, skin soothing agents, skin healing agents and skin conditioning.

The present invention provides a suspension of SC extract compounded with a steroid ingredient and/or NSAID for pharmaceutical purposes as described above. The concentration of the active compound in pharmaceutical formulation will vary based on the condition being treated.

One example of a pharmaceutical formulation of the present invention would include a topical pharmaceutical gel formulated with the 0.01% to 10% active compound, isopropyl alcohol, hydroxyethylcellulose, citric acid monohydrate, sodium citrate dihydrate, benzyl alcohol and purified water.

In one embodiment of the invention, a gel formulation may be used for the treatment of actinic keratoses on sun damaged skin. More specifically, the invention might be applied on the affected skin as a single dose or with repeated dosing. Dosing regimens would vary based on the condition being treated.

In another embodiment of the invention, a suspension form is contemplated for use in the treatment of basal cell carcinoma, squamous cell carcinoma, and other types of skin cancer. A suspension form is also contemplated for use in the treatment of benign cutaneous neoplasms (e.g., seborrheic keratosis), photodamaged skin (wrinkles, fine lines, lentigines), wound healing (scar reduction/minimization), and skin infections (e.g., human papilloma virus infections).

1. A topical medicament (ointment/gel/cream/lotion/solution) that consists of SC 0.01%-10%, a steroid 0.01%-10%, and/or a non-steroidal anti-inflammatory medication 0.01%-10% with other ingredients that is used to treat benign cutaneous neoplasms (e.g. seborrheic keratoses, condyloma, verucca), precancerous lesions (e.g. actinic keratoses), photodamaged skin, and non-melanoma skin cancers (e.g. squamous cell carcinoma, basal cell carcinoma).

2. The method of removing and treating benign cutaneous neoplasms, precancerous lesions, photo-damaged skin, and non-melanoma skin cancers using the topical medicament in claim 1, wherein the medicament is administered as a single application or applied a plurality of times.
